(19) United States
(12) Patent Application Publication
(10) Pub. No.: US 2008/0167607 A1
(43) Pub. Date: Jul. 10, 2008

(54) Enteral Feeding Catheter, Computer System and Computer Program for Operating the Feeding Catheter

(76) Inventors: Ulrich Pfeiffer, Munich (DE); Reinhold Knoll, Munich (DE); Manu Malbrain, Lovenjoel (BE)

Correspondence Address: COLLARD & ROE, P.C., 1077 NORTHERN BOULEVARD, ROSLYN, NY, 11576, US

(21) Appl. No.: 11/884,994

(22) PCT Filed: Jan. 3, 2006
(86) PCT No.: PCT/EP06/50009

§ 371 Date: Nov. 14, 2007

(30) Foreign Application Priority Data
Feb. 25, 2005 (EP) 05101465.2

(51) Int. Cl. A61M 25/10

(52) U.S. Cl. 604/97.01; 604/101.05; 604/99.01

(57) ABSTRACT

The invention relates to an enteral feeding catheter (1) for channelling flowable nutrient into a patient's digestive tract, such as into patients stomach, duodenum or jejunum, said catheter having a first lumen (2), having an opening (3) for connecting it to a supply for of nutrient and channelling said nutrient from the proximal end (P) to the distal end (D), the distal end (D) of the first lumen (2) being provided with several radial outlets (4) for the exit of said nutrient into patient's digestive tract,

the catheter (1) further comprising a second lumen (7), for being connected to a source/sink of gas (26) and a gas pressure gauge (24) at its proximal end and being connected to an inflatable and deflatable distal balloon (6) provided on the catheter (1) near the distal end (D).

The invention refers also to a computer system and computer program to operate said feeding catheter.

The invention relates to an enteral feeding catheter for channelling flowable nutrient into a patient's digestive tract, such as into patient's stomach, duodenum or jejunum, said catheter being adapted to be advanced through patient's esophagus, said catheter having a proximal end, a distal end, and a first lumen, said first lumen having an opening at said proximal end adapted to be connected to a supply for receiving said nutrient from said supply and channelling said nutrient from said proximal end to said distal end, said distal end being provided with several radial outlets for the exit of said nutrient.

Feeding catheters of this type are used for instance in feeding of a patient who is unable to swallow nutrients in the natural manner or in postoperative or critical care. Especially in critical care it is important to monitor the intra-abdominal pressure in order to avoid intra-abdominal hypertension or abdominal compartment syndrome, which may impede blood circulation and perfusion in the abdominal region and may be hazardous to the patient.

DE 35 00 822 A1 discloses a device for measuring the pressure in the human or animal body. The device comprises a balloon which is attached at the distal end of a multi-luminal catheter and which is only partially filled with a gaseous medium. The pressure in the balloon is transferred over a lumen of the catheter to a pressure gauge. DE 35 00 822 A1 does not disclose an enteral feeding catheter.

It is therefore an object of the invention to provide an enteral feeding catheter device which allows reliable continuous determination of patient's intra-abdominal pressure and keeps the additional burden related to the pressure measurement for the critical ill patient as low as possible.

It is a further object of the invention to provide a computer system and a computer program which allow for calibration and recalibration of the measurement equipment and allow continuous determination of the intra-abdominal pressure with good accuracy.

According to the invention an enteral feeding catheter as mentioned above is provided with a second lumen, adapted to be connected to a source/sink of gas and a gas pressure gauge at its proximal end and being connected to an inflatable and deflatable distal balloon provided on said catheter body near said distal end. The balloon will be named “distal balloon” in the following just for the sake to give it a name and to distinguish it from other balloons (described later) which are situated proximally with respect to the distal balloon. “Distal” must not be understood in the sense that the distal balloon is situated at the very distal end of the catheter body.

The distal balloon has preferably a high degree of gastightness allowing relatively long recalibration intervals.

Preferably the enteral feeding catheter has a third lumen adapted to be connected to a source/sink of air and a gas pressure gauge at its proximal end and being connected to an inflatable and deflatable proximal balloon provided on said catheter body at an axial, position such that said second balloon is positioned in the patient's thorax when the catheter is inserted. The balloon will be named “proximal balloon” in the following just for the sake to give it a name and to distinguish it from the distal balloon which is situated distally with respect to the proximal balloon. “Proximal” must not be understood in the sense that the balloon is situated close to the proximal end of the catheter body. Providing the catheter with a proximal balloon which is situated in the thorax region of the patient, i.e. above the diaphragm when the catheter is properly inserted in place, allows to monitor not only the intra-abdominal pressure but also the intra-thoracic pressure which has an importance in monitoring cardiovascular parameters of the patient which cannot be overestimated.

According to another preferred embodiment of the invention the enteral feeding catheter has a fourth lumen adapted to be connected to a source/sink of gas or fluid at its proximal end and being connected to a blocking balloon provided on said catheter body at an axial position such that said blocking balloon is positioned in patients stomach when the catheter is inserted. This blocking balloon, which is preferably filled with air or water, is very useful to assist in placing the catheter precisely and correctly without applying x-ray imaging. The catheter is advanced through the patient's esophagus with the blocking balloon being empty until the operator can be sure that the blocking balloon has entered the stomach. The balloon is then filled with air or water and the catheter is withdrawn until the blocking balloon abuts the cardia of patient's stomach, thus ensuring that the catheter is placed precisely with respect to the stomach. Additionally the blocking balloon prevents the catheter from being withdrawn further from its place. In order to avoid accidental overpressure the pressure inside the balloon could be limited and/or monitored. An overpressure would indicate falsely positioning of the blocking balloon in the esophagus.

Alternatively or additionally the feeding catheter, may be provided with several radioopaque marking rings distributed over its length and/or nearby the balloons and/or a radioopaque stripe extending axially over its length to allow observing the placement of the catheter under X-ray-imaging.

Insertion marks distributed over the length of the catheter may inform the operator on how far the catheter has been advanced into the patient's body.

Additionally the feeding catheter ray be provided with two or more electrodes placed near the distal end of the catheter for impedance measurement to determine the acidity of the gastric juice and to carry out gastric volume measurements. The gastric volume is negative correlated with the bio-electrical impedance i.e. impedance decreases if gastric volume increases. The gastric volume could be calculated with a empirical estimated function of the impedance. Additionally or alternatively the feeding catheter may provide two or more electrodes sufficiently proximal before or after the proximal balloon 8. These electrodes should be placed above diaphragm in the esophagus in order to estimate transthoracic bioimpedance which measures cardiac stroke volume and cardiac output and thoracic fluid content. Additionally the feeding catheter may provide pulse densitometry means placed in the esophagus in order to measure arterial oxygen saturation or indo cyanine green dye concentration. It was found that this would be more reliable than common non invasive pulse densitometry means e.g. on the finger or ear.

A computer system which provides for calibration and recalibration of the measurement equipment and allows continuous determination of the intra-abdominal pressure with good accuracy comprises first connection means adapted to connect said computer system to pump driving means adapted to drive a pump for increasing/decreasing the gas volume inside at least one balloon of an enteral feeding catheter and second connection means adapted to connect said computer system to a pressure gauge adapted to determine the pressure p prevailing in said at least one balloon, calculation means for calculating the mathematical derivative dp/dV of pressure p with respect to volume V displaced by the pump and accessing means to access executable instructions to cause said computer system to cause said pump driving means to adjust the gas volume inside said balloon for initial setting such that said mathematical derivative dp/dV of pressure p with respect to volume V is zero or as close to zero as possible.

Preferably the computer system further comprises third connection means adapted to connect said computer system to valve means adapted to connect for initial settings said pressure gauge to ambient air and disconnect it from ambient air, said accessing means access instructions to cause said computer system to connect said pressure gauge to ambient air and to adjust said pressure gauge to zero.

Advantageously the computer system further comprises alarm means alerting of a blocked or occluded catheter lumen, in case that the absolute mathematical derivative dp/dV of pressure p with respect to volume exceeds a certain upper threshold at a certain pressure.

The computer system may further comprise alarm means alerting of a leakage in case that the absolute mathematical derivative dp/dV of pressure p with respect to volume V fails to reach a lower threshold at a certain pressure.

A computer program which provides for calibration and recalibration of the measurement equipment and allows continuous determination of the intra-abdominal pressure with good accuracy comprises instructions executable by a computer system to cause said computer system for initial settings to drive a pump for increasing/decreasing the gas volume inside at least one balloon of said enteral feeding catheter and collecting pressure readings of pressure p prevailing in said at least one balloon, calculating the mathematical derivative dp/dV of pressure p with respect to volume V and adjusting the gas volume inside said balloon such that said mathematical derivative dp/dV of pressure p with respect to volume V is zero or as close to zero as possible.

Preferably the computer program comprises the steps of connecting said pressure gauge to ambient air, adjusting said pressure gauge to zero and disconnecting said pressure gauge from ambient air.

Advantageously the computer program comprises the step of activating alarm means alerting of a blocked or occluded catheter lumen, in case that the absolute mathematical derivative dp/dV of pressure p with respect to volume V exceeds a certain upper threshold at a certain pressure.

Preferably the computer program further comprises the step of activating alarm means alerting of a leakage in a catheter lumen, in case that the absolute mathematical derivative dp/dV of pressure p with respect to volume V fails to reach a lower threshold at a certain pressure.

Preferably the computer program further comprises the steps of continuously collecting pressure readings after initialization has been accomplished.

Preferably the computer program further comprises the step of activating an alarm when the pressure exceeds a certain threshold.

The invention including its construction and method of operation will be illustrated in the drawings in which

FIG. 1 is an overall view of a preferred embodiment of the feeding catheter according to the invention;

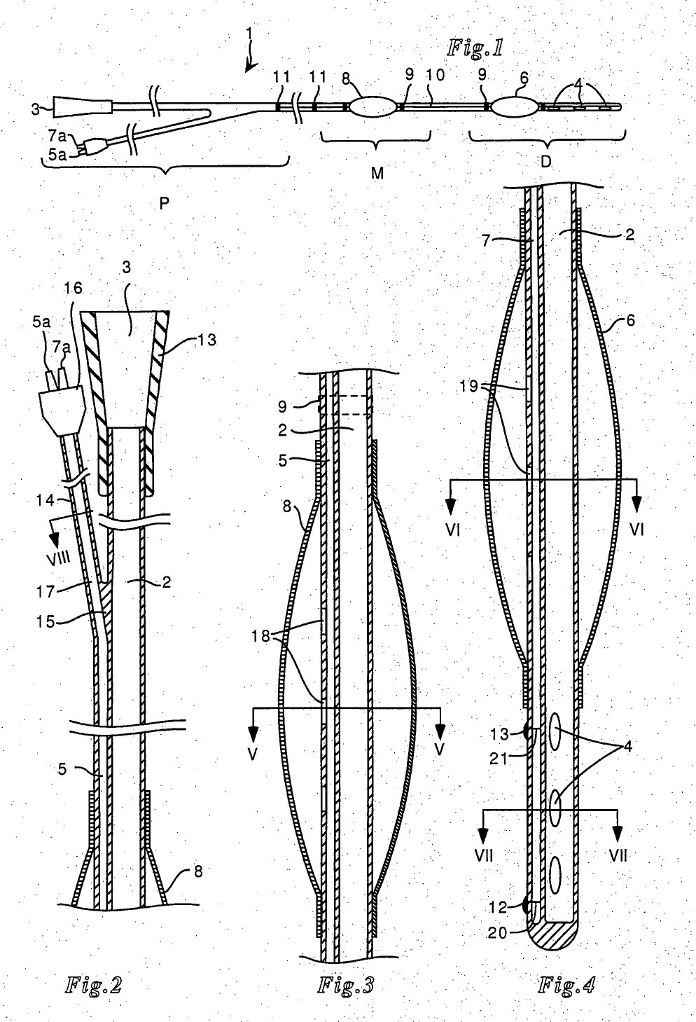

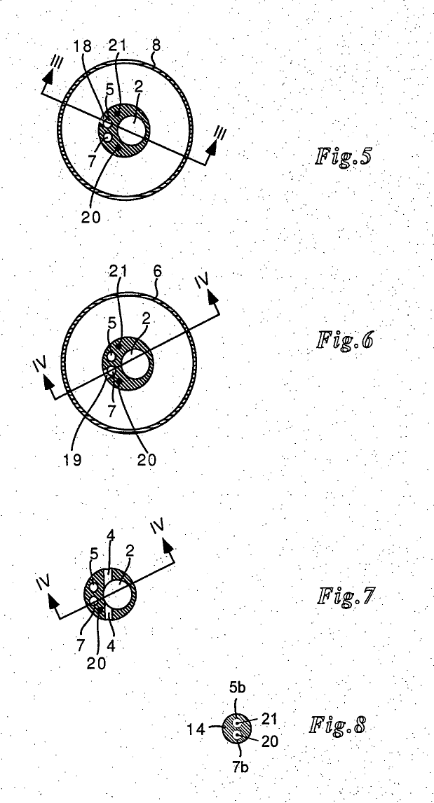

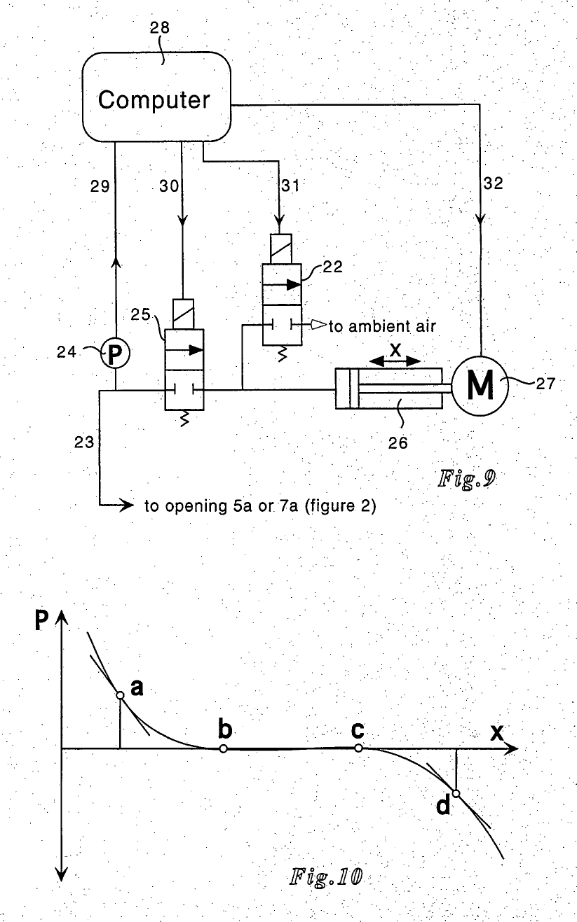

FIG. 2 is a longitudinal sectional view of the proximal part of the catheter of FIG. 1;

FIG. 3 is a longitudinal, sectional view of the middle part of the catheter of FIG. 1 taken along line III-III of FIG. 5;

FIG. 4 is a longitudinal sectional view of the distal part of the catheter of FIG. 1 taken along line IV-IV of FIG. 6;

FIG. 5 is a cross sectional view of the catheter taken along line V-V in FIG. 3;

FIG. 6 is a cross sectional view of the catheter taken along line VI-VI in FIG. 6;

FIG. 7 is a cross sectional view of the catheter taken along line VII-VII in FIG. 4;

FIG. 8 is a cross sectional view of the catheter taken along line VIII in FIG. 2;

FIG. 9 is a diagrammatic view of a computerized system for operating the catheter of the invention;

FIG. 10 is a diagram showing the pressure P measured with a pressure gauge as a function of the displacement x of a piston;

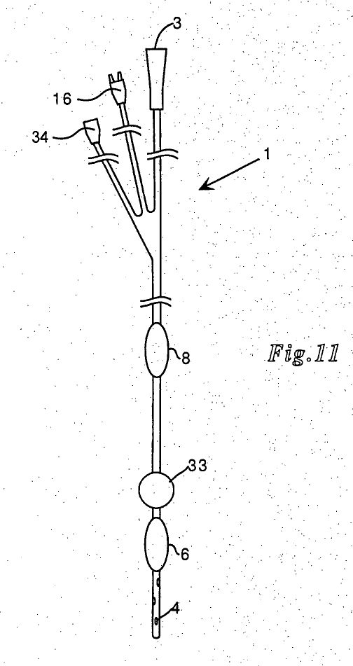

FIG. 11 is an overall view of a further embodiment of the feeding catheter according to the invention.

FIG. 1 shows a preferred embodiment of the feeding catheter according to the invention. The catheter 1 has three sections, namely a proximal section P, a middle section M and a distal section D. The catheter has at its proximal end an opening 3 by means of which which it may be connected to a supply of nutrient, which may be hanged at a infusion stand (not shown). There are two further openings, namely openings 5a and 7a, to which sources/sinks of gas pressure may be connected.

In the middle part M the body of the catheter 1 is surrounded by a proximal balloon 8 which is internally connected to the opening 5a, so that the proximal balloon 8 may be inflated by pressing air into the opening 5a or may be deflated by sucking air out of opening 5a which will be explained in detail later. The balloon 8 is named “proximal balloon” just for the sake to give it a name and to distinguish it from a balloon which is situated distally with respect to the proximal balloon which will be described later. “Proximal” is not to be understood that the balloon is situated at the proximal end of the catheter body.

In the distal part D the body of the catheter 1 is surrounded by a distal balloon 6 which is internally connected to the opening 7a, so that the distal balloon 6 may be inflated by pressing air into the opening 7a or may be deflated by sucking air out of the opening 7a which will be explained in detail later. The balloon 6 is named “distal balloon” just for the sake to give it a name and to distinguish it from the proximal balloon 8 which is situated proximally with respect to the distal balloon 6 mentioned before. “Distal” must not be understood that the balloon is situated at the very distal end of the catheter body.

Distally with respect to the distal balloon 6 there are provided radial openings 4 which serve as outlets to allow flowable nutrients or medications, which have been introduced into the catheter 1 through its opening 3, to exit into patient's digestive tract.

Before and after balloons the catheter has radio opaque marking rings 9, an axially extending radio opaque stripe 10, and optically visible insertion marks 11, supporting the operator in determining the position of the catheter in patient's body, especially during the time when it is advanced through the digestive tract of the patient. The marks 10 extent from balloon 8 up to the Y-junction 15 at equal distances (e.g. 5 cm).

The details of the structure of catheter 1 will become more apparent from FIG. 1 which shows a longitudinal section of the proximal part of the catheter 1. The proximal opening 3 of the catheter 1 is provided with a conical connector 13 to which a supply line for nutrients may be connected. The supply line (not shown) will disconnect from the connector 13 automatically if an excessive pulling force is exerted thereby protecting the patient from being injured. Downstream or distally of the connector 13 there is a first lumen 2, which serves to guide flowable nutrients or medicine down into patient's digestive tract.

A side hose 14 is connected to the main part of the catheter 1 via a Y-junction 15. The distal end of the side hose 14 is closed by a connector 16, which has two openings 5a and 7a. The side hose 14 is connected to a second lumen 5 and a third lumen which is not visible in FIG. 2. Inside the side hose 14 there is provided a lumen 5b which connects the opening 5a to the second lumen 5. There is a further lumen 7b in the side hose 14, which connects the second opening 7a to the third lumen 7 which is however not visible in the sectional view of FIG. 2. Downstream of the Y-junction 15 the second lumen 5 is parallel to the larger first lumen 2.

As may be best understood from FIG. 3 the lumen 5 is connected to the proximal balloon 8. In the region where the body of the catheter 1 is surrounded by the proximal balloon 8 the outer wall of the second lumen 5 has radial openings 18 which allow for gas flow between the second lumen 5 and the proximal balloon 8. As a result the interior of the proximal balloon 8 is connected to the opening 5a (FIG. 2) so that the proximal balloon 8 can be inflated by blowing air or another gas into the opening 5a.

FIG. 4 shows the distal part D of the catheter 1. It is to be observed that—although the drawing of FIG. 4 looks similar to that of FIG. 3—the plane of section is different to that of FIG. 3, which can be best understood from FIGS. 5 and 6 in which the respective planes of section are indicated by arrows marked with “III” and “IV”. As can be seen the plane of section for FIG. 3 traverses the second lumen 5 diametrally, while the plane of section of FIG. 4 traverses the third lumen 7 diametrally.

As becomes apparent from FIG. 4, the third lumen 7 is connected to the interior of the distal balloon 6 via radial openings 19. The interior of the distal balloon 6 communicates with the opening 7a and can hence be inflated and deflated through that opening 7a. The surface inside the balloons 6 and 8 is roughened or profiled in a way to ensure complete emptying of gas at deflation.

Close to the distal tip of the catheter 1 the first lumen 2 is provided with several radial outlets 4 which allow nutrient to flow out of the first lumen 2. At the outer wall of the catheter 1 there are further provided two electrodes 12 and 13 spaced apart from each other. The electrodes are connected by electric wires 20 and 21 to the connector 16 (FIG. 2).

FIG. 5 is a cross sectional view of the catheter taken along line V-V in FIG. 3. FIG. 5 shows the catheter with its first-lumen 2, second lumen 5, third lumen 7 and embedded connection wires 20, 21 for the electrodes 12, 13 shown in FIG. 4. Alternatively the wires 20 and 21 could be placed inside the lumina 5 and 7. The second lumen 5 is connected via openings 18 to the interior of the proximal balloon 8, while there is no such connection between the third lumen 7 and proximal balloon 8.

FIG. 6 is a cross sectional view of the catheter taken along line IV-IV in FIG. 4. FIG. 6 shows the catheter with its first lumen 2, second lumen 5, third lumen 7 and connection wires 20, 21 for the electrodes 12, 13 shown in FIG. 4. The third lumen 7 is connected via openings 19 to the interior of the distal balloon 6, while there is no such connection 20 between the second lumen 5 and the distal balloon 6.

FIG. 7 is a cross sectional view of the catheter taken along line VII-VII in FIG. 4. FIG. 7 shows, the catheter with its first lumen 2, second lumen 5, third lumen 7 and connection wire 20 for the electrode 12 shown in FIG. 4. The first lumen 2 has openings 4 allowing nutrient to flow out into patient's digestive tract.

FIG. 8 is a cross sectional view of the catheter taken along line VIII-VIII in FIG. 2. FIG. 2 shows the side hose 14 which is connected to the main part of the catheter 1 via a Y-junction 15 (FIG. 2). Inside the side hose 14 there are is provided the lumen 5b which connects the opening 5a to the second lumen 5 (FIG. 2). There is a further lumen 7b in the side hose 14, which connects the second opening 7a (FIG. 2) to the third lumen 7 (FIG. 4).

FIG. 9 is a diagrammatic view of a computerized system for operating the catheter 1 of the invention. The catheter 1 is connected via its opening 5a to air conduit 23. The air conduit 23 is connected to a pressure gauge 24, by which the pressure in the air conduit 23 can be measured continuously. The air conduit 23 is further connected over a first electromagnetic valve 25 to a piston pump 26 driven by a motor 27. The air conduit 23 is further connected to a second electromagnetic valve 22, by which the conduit may be connected to ambient air. A computer 28 is electrically connected via line 29 to the pressure gauge 24, via line 30 to the first electromagnetic valve 25, via line 31 to the second electromagnetic valve 22 and via line 32 to the electric motor 27. By means of these connections the computer 28 is able to collect pressure readings from the pressure gauge 24, to open and close the first and second valves 25 and 22 and to activate the motor 27 in order to advance or retract the piston of the piston pump 26. The catheter 1 may be connected via its opening 7a to an identical system or a switch valve (not shown) may be provided to connect the system shown in FIG. 9 selectively to the opening 5a and 7a of the catheter 1.

The catheter system is operated as follows:

The catheter 1 is advanced with its distal tip ahead through the mouth or the nose of a patient into the esophagus of the patient until its tip reaches the stomach, the duodenum or the jejunum of the patient depending on the medical requirements. The axial position of the distal balloon is such that the distal balloon will be placed in the stomach (or duodenum or jejunum), while the axial position of the proximal balloon on the body of the catheter 1 is such that it will be placed in the patient's thorax. The movement of the catheter 1 is monitored by means of the radioopaque marking rings 9, radioopaque stripe 10 and/or by counting visible insertion marks 11 outside the patient.

Once the distal tip of the catheter 1 has reached its final destination in the patient's digestive tract, the computerized system as shown in FIG. 9 is connected to the opening 5a. The computer program that runs on the computer 28 causes the first and the second electromagnetic valves 22 and 25 to open, so that the air conduit 23 is connected to ambient air. Then the piston 26 is moved to a defined e.g. middle position and pressure gauge 24 is set to zero. The computer 28 then causes the electromagnetic valve 22 to close in order to disconnect the air conduit 23 from ambient air. The computer causes the motor 27 to drive the piston of piston pump 26 in forward direction so that air is blown into the proximal balloon 8 via air conduit 23, opening 5a, lumen 5b, lumen 5 and openings 18, while taking pressure readings from the pressure gauge 24 and calculating the mathematical derivative dp/dV of pressure p with respect to volume V permanently. Instead of calculating dp/dV equally dp/dx may be calculated where x is the position of the piston of the piston pump 26. The curves dp/dV and dp/dx have the same characteristic form and differ only by a constant scaling factor which corresponds to the (constant) cross section of the piston. The same applies for variables x and V, so that it does not make any difference if V or x is recorded.

During the inflation process the pressure inside the proximal balloon 8 increases and the balloon 8 unfolds. After the balloon 8 has been unfolded the pressure inside the balloon increases rapidly, in other words the absolute amount of mathematical derivative of pressure p with respect to displacement x of the piston is high and exceeds a predetermined threshold at a certain positive pressure, causing the program to stop the inflation process and to start a deflation in other words the piston will be moved in the opposite direction whereby the balloon 8 is evacuated. At the beginning of the deflation process the pressure falls rapidly until the balloon 8 begins to collapse. During the process of collapsing the balloon 8 the pressure changes only slightly or remains constant, in other words dp/dV and equally dp/dx are zero or at least very close to zero. When the balloon 8 has reached the state of being completely collapsed further movement of the piston will cause rapid pressure drop, in other words dp/dV and dp/dx respectively will have large absolute amounts. When the absolute amount of the derivative with respect to volume exceeds a predetermined threshold at a certain negative pressure the evacuation action will be stopped by the computer 28.

The diagram shown in FIG. 10 reflects the evacuation process. At the beginning (point a) the pressure drops rapidly and remains approximately constant between points b and c. Further evacuation, i.e. further withdrawal of the piston of the piston pump 26 causes an increasingly rapid pressure drop until point d is reached.

The pressure inside the stomach can however be assessed reliably when it is independent of the filling state of balloon 8, i.e. in the range between point b and point c.

After having collected the pressure readings and having calculated the derivative dp/dx (or dp/dV) the computer determines a position between points b and d preferably the middle between points b and d, and moves the piston to this position to finalize the initial setting of the system.

In order to minimize leakage, valve 25 is closed after this calibration phase. If leakage is negligible valve 25 could be omitted. Then the patients thoracic pressure can be assessed continuously. After some time due to inavoidable leakage e.g. of the balloon a recalibration may become indicated.

Assumed now that the balloon 8 or any other part as for example the connector 16 has a leakage. In this case the pressure p and its mathematical derivative dp/dV with respect to volume V will remain low during the process of inflation and the pressure and its derivative will remain below a predetermined threshold. This will be recognized by the computer system and an alarm will be given.

Further it may occur that a lumen may be occluded or intentionally blocked because the catheter used has only one balloon. This situation as well will be recognized by the computer system because in this case the pressure during the inflation process will go high very rapidly.

Needless to say that the initial adjustment for the distal balloon 6 is done in the same way; by connecting the opening 7a to the computerized system and proceeding in the same way as described above with respect to the proximal balloon 8 in order to determine the patient's intra abdominal pressure. The computer may then calculate also the difference between abdominal and thoracal pressure and monitor this difference value.

While an embodiment has been described in which a catheter with two balloons is employed it goes without saying that only one balloon may be employed if the measurement of the pressure at only one site in patient's body is regarded to be sufficient. One of the two balloons and the catheter lumen pertaining to it may be left away.

FIG. 11 is an overall view of a further embodiment of the feeding catheter according to the invention. The catheter has a proximal balloon 8 and a distal balloon 6 and is constructed in the same way as the catheter described with reference to the drawings 1 to 8. However, the catheter is provided with an additional balloon 33 which serves as a blocking balloon. The balloon 33 is connected to an additional lumen (not shown in the drawing) in the same way as described with regard to balloons 6 and 8 of the first embodiment. In the embodiment of FIG. 11 the additional connector 34 is provided with one further opening allowing to supply a liquid under pressure into the balloon 33 and to withdraw it from the balloon 33.

The catheter of FIG. 11 is applied as follows:

The catheter 1 is advanced with its distal tip ahead through the mouth or the nose of a patient into the esophagus of the patient until its tip reaches the stomach, the duodenum or the jejunum of the patient depending on the medical requirements. The axial position of the distal balloon 6 is such that the distal balloon 6 as well as the blocking balloon 33 will then be situated in the stomach. The movement of the catheter 1 is monitored by means of counting the visible insertion marks 11 to make sure that the blocking balloon has entered the stomach. Then the blocking balloon 33 is filled with water and the catheter will be retracted until the blocking balloon 33 abuts the cardia of the patient's stomach, thus ensuring that the catheter is placed precisely with respect to the stomach and balloon 8 will be situated in the patient's thorax. Additionally the blocking balloon prevents the catheter 1 from being withdrawn from its place. This blocking balloon is very useful to assist in placing the catheter precisely and correctly without applying X-ray imaging.

The further operation of the catheter 1 is same as described for the catheter of the first embodiment and may be omitted therefore.

1. Enteral feeding catheter for channelling flowable nutrient into a patient's digestive tract, such as into patient's stomach, duodenum or jejunum, said catheter being adapted to be advanced through patient's esophagus,
said catheter having a proximal end, a distal end, and a first lumen,
said first lumen having an opening at said proximal end adapted to be connected to a supply for receiving said nutrient from said supply and channelling said nutrient from said proximal end to said distal end,
said distal end of said first lumen being provided with at least one outlet for the exit of said nutrient into patient's digestive tract,
said catheter further comprising a second lumen, adapted to be connected to a source/sink of gas and a gas pressure gauge at its proximal end and being connected to an inflatable and deflatable distal balloon provided on said catheter near said distal end.

2. Enteral feeding catheter as claimed in claim 1, wherein said distal balloon is gastight.

3. Enteral feeding catheter as claimed in claim 1, further comprising a third lumen adapted to be connected to a source/sink of gas and a gas pressure gauge at its proximal end and being connected to an inflatable and deflatable proximal balloon provided on said catheter at an axial position such that said proximal balloon is positioned in patient's thorax when the catheter is inserted.

4. Enteral feeding catheter as claimed in claim 1, further comprising a further lumen adapted to be connected to a source/sink of fluid at its proximal end and being connected to a blocking balloon provided on said catheter at an axial position such that said blocking balloon is positioned in patient's stomach when the catheter is inserted.

5. Enteral feeding catheter as claimed in claim 1, further comprising several radioopaque marking rings distributed over its length.

6. Enteral feeding catheter as claimed in claim 1, further comprising a radioopaque stripe extended over its length.

7. Enteral feeding catheter as claimed in claim 1, further comprising several insertion marks distributed over its length.

8. Enteral feeding catheter as claimed in claim 1, further comprising at least two electrodes placed near the distal end of the catheter for impedance measurement and gastric volume measurement.

9. Computer system comprising first connection means adapted to connect said computer system to pump driving means adapted to drive a pump for increasing/decreasing the gas volume V inside at least one balloon of an enteral feeding catheter as claimed in claim 1, and second connection means adapted to connect said computer system to a pressure gauge adapted to determine the pressure p prevailing in said at least one balloon, calculation means for calculating the mathematical derivative dp/dV of pressure p with respect to volume V and accessing means to access executable instructions to cause said computer system to cause said pump driving means to adjust the gas volume inside said balloon for initial setting such that said mathematical derivative dp/dV of pressure p with respect to volume V is zero or as close to zero as possible.

10. Computer system as claimed in claim 9, further comprising third connection means adapted to connect said computer system to valve means adapted to connect for initial settings said pressure gauge to ambient air and disconnect it from ambient air, said accessing means access instructions to cause said computer system to connect said pressure gauge to ambient air and to adjust said pressure gauge to zero.

11. Computer system as claimed in claim 9, further comprising alarm means alerting of a blocked or occluded catheter lumen, in case that the mathematical derivative dp/dV of pressure p with respect to volume V exceeds a certain upper threshold at a certain pressure.

12. Computer system as claimed in claim 9, further comprising alarm means alerting of a leakage in case that the mathematical derivative dp/dV of pressure p with respect to volume V fails to reach a lower threshold at a certain pressure.

13. Computer program, especially computer program stored on a machine-readable storage medium, for carrying out pressure measurements in an enteral feeding catheter as claimed in claim 1, comprising instructions executable by a computer system to cause said computer system for initial settings to drive a pump for increasing/decreasing the gas volume V inside at least one balloon of said enteral feeding catheter and collecting pressure readings of pressure p prevailing in said at least one balloon, calculating the mathematical derivative dp/dV of pressure p with respect to volume V and adjusting the gas volume inside said balloon such that said mathematical derivative dp/dV of pressure p with respect to volume V is zero or as close to zero as possible.

14. Computer program as claimed in claim 13, further comprising the steps of connecting said pressure gauge to ambient air, adjusting said pressure gauge to zero and disconnecting said pressure gauge from ambient air.

15. Computer program as claimed in claim 13, further comprising the step of activating alarm means alerting of a blocked or occluded catheter lumen, in case that the mathematical derivative dp/dV of pressure p with respect to volume V exceeds a certain upper threshold at a certain pressure.

16. Computer program as claimed in claim 13, further comprising the step of activating alarm means alerting of a leakage in a catheter lumen, in case that the mathematical derivative dp/dV of pressure p with respect to volume V fails to reach a lower threshold at a certain pressure.

17. Computer program as claimed in claim 13, further comprising the steps of continuously collecting pressure readings after initialization has been accomplished.

18. Computer program as claimed in claim 17, further comprising the step of activating an alarm when the pressure exceeds a certain threshold.

19. Method of carrying out pressure measurements comprising the steps of
(a) providing an enteral feeding catheter having a proximal end, a distal end, and a first lumen, said first lumen having an opening at said proximal end and at least one outlet for the exit of a nutrient into a patient's digestive tract at said distal end, said enteral feeding catheter further comprising a second lumen being connected to an inflatable and deflatable distal balloon provided on said catheter near said distal end,
(b) connecting the opening of said first lumen at said proximal end of said enteral feeding catheter to a supply of said nutrient for receiving said nutrient from said supply and for channelling said nutrient from said proximal end to said distal end,
(c) connecting said second lumen to a source/sink of gas and a gas pressure gauge,
(d) advancing said enteral feeding catheter through the patient's esophagus,
(e) increasing/decreasing the gas volume V inside said balloon of said enteral feeding catheter
(f) collecting pressure readings of pressure p prevailing in said distal balloon,
(g) calculating the mathematical derivative dp/dV of pressure p with respect to volume V and (e) adjusting the gas volume inside said distal balloon such that said mathematical derivative dp/dV of pressure p with respect to volume V is zero or as close to zero as possible.

20. Method as claimed in claim 19, further comprising the steps of connecting said pressure gauge to ambient air, adjusting said pressure gauge to zero and disconnecting said pressure gauge from ambient air.

21. Method as claimed in claim 19, further comprising the step of alerting of a blocked or occluded catheter lumen, in case that the mathematical derivative dp/dV of pressure p with respect to volume V exceeds a certain upper threshold at a certain pressure.

22. Method as claimed in claim 19, further comprising the step of alerting of a leakage in a catheter lumen, in case that the mathematical derivative dp/dV of pressure p with respect to volume V fails to reach a lower threshold at a certain pressure.

23. Method as claimed in claim 19, further comprising the steps of continuously collecting pressure readings after initialization has been accomplished.

24. Method as claimed in claim 23, further comprising the step of activating an alarm when the pressure exceeds a certain threshold.

25. Method as claimed in claim 19, wherein said enteral feeding catheter is provided having an inflatable and deflatable proximal balloon at an axial position such that said proximal balloon is positioned in patient's thorax when the catheter is inserted, and a third lumen connected to said proximal balloon, said third lumen having a proximal end, and wherein said method further comprises the steps of connecting said third lumen to a source/sink of gas and a gas pressure gauge at its proximal end, and
increasing/decreasing the gas volume inside said proximal balloon of said enteral feeding catheter
collecting pressure readings of pressure prevailing in said proximal balloon,
calculating the mathematical derivative of pressure in said proximal balloon with respect to volume and
adjusting the gas volume inside said proximal balloon such that said mathematical derivative of pressure in said proximal balloon with respect to volume is zero or as close to zero as possible.

26. Method as claimed in claim 19, wherein said enteral feeding catheter is provided having a blocking balloon provided on said catheter at an axial position such that said blocking balloon is positioned in patient's stomach when the catheter is inserted, and a further lumen connected to said blocking balloon, said further lumen having a proximal end, and wherein said method further comprises the step of connecting said further lumen to a source/sink of fluid at its proximal end.
